(19) United States
(12) Patent Application Publication
(10) Pub. No.: US 2025/0099670 A1
(43) Pub. Date: Mar. 27, 2025

(54) NASOGASTRIC TUBE

(71) Applicant: CAMBRIDGE UNIVERSITY HOSPITALS NHS FOUNDATION TRUST, Cambridge, Cambridgeshire (GB)

(72) Inventor: Andrew Hindmarsh, Cambridge (GB)

(73) Assignee: CAMBRIDGE UNIV. HOSPITALS NHS FOUNDATION TRUST, Cambridge, Cambridgeshire, GB (GB)

(21) Appl. No.: 18/539,357

(22) PCT Filed: Jun. 24, 2022
(86) PCT No.: PCT/GB2022/051632

§ 371 Date: Dec. 14, 2023

(30) Foreign Application Priority Data
Jun. 24, 2021 (GB) 2109097.2

(51) Int. Cl. A61M 1/00

(52) U.S. Cl. CPC A61M 1/90; A61M 2205/0266

(57) ABSTRACT

A nasogastric tube comprising a porous medium for treatment of a treatment area of an internal surface of a body lumen of a patient to be treated; a suction lumen coupled to the porous medium for providing suction to the porous medium; and a radial deployment system operable to deploy the porous medium between: (i) an insertion state for insertion of the porous medium into the patient, and (ii) a treatment state in which an outer surface of the porous medium is moved towards the treatment area of the patient; wherein a window is defined in a body carried by the nasogastric tube; and wherein a portion of the radial deployment system and/or a portion of the porous medium is arranged to move radially outwards through the window to deploy the porous medium from the insertion state to the treatment state.

### TECHNICAL FIELD

The present disclosure relates to devices and methods for treatment of an internal surface of a human or animal body.

### BACKGROUND

Nasogastric tubes can be inserted through a patient's nasal passageway and used to provide access to their GI tract. Nasogastric tubes can be passed into the patient's stomach to provide short to medium term nutritional support, or to provide aspiration of stomach contents. For example, a nasogastric tube may be used to provide fluid drainage to help facilitate recovery from issues associated with the bowel, such as a bowel obstruction. Typically, nasogastric tubes are made of a polymer material which can withstand gastric acid for 2 to 6 weeks (depending on the type of material used).

WO 2017/182827 A1 discloses devices and methods for treatment of internal defects of a human or animal body. It discloses a catheter including a tube, an applicator and a porous medium, wherein the applicator can be controlled at a proximal end of the tube to deploy the porous medium from a distal end of the tube to treat the defect.

### SUMMARY

Aspects of the disclosure are set out in the independent claims and optional features are set out in the dependent claims. Aspects of the disclosure may be provided in conjunction with each other, and features of one aspect may be applied to other aspects.

In an aspect, there is provided a nasogastric tube comprising: a porous medium for treatment of a treatment area of an internal surface of a body lumen of a patient to be treated; a suction lumen coupled to the porous medium for providing suction to the porous medium; and a radial deployment system operable to deploy the porous medium between: (i) an insertion state for insertion of the porous medium into the patient, and (ii) a treatment state in which an outer surface of the porous medium is moved towards the treatment area of the patient. A window is defined in a body carried by the nasogastric tube. A portion of the radial deployment system and/or a portion of the porous medium is arranged to move radially outwards through the window to deploy the porous medium from the insertion state to the treatment state.

Embodiments may enable radial deployment of porous medium from a position radially inward of a treatment area in an internal surface of a body lumen of a patient. The nasogastric tube may be positioned in the lumen with the porous medium radially inward of, and aligned with, a portion of the treatment area. The porous medium may then be deployed radially outward so that an outer surface of the porous medium is contact with, or at least radially closer to, the porous medium. Thus, the porous medium may be aligned with the treatment area prior to deployment. The deployment may therefore lead to accurate placement of the porous medium, as the porous medium will have deployed radially outwards from a selected location inside the treatment area.

The window may be defined in a body of the nasogastric tube (e.g. an outer surface of the tube). The window may comprise an opening, such as an aperture, in a surface of a body of the nasogastric tube. For example, the window may comprise an aperture in a tangential surface of the body. For example, the nasogastric tube may comprise a housing (e.g. which houses components of the tube), and the window may be an aperture in a tangential surface of the housing. The outer surface of the porous medium may be expanded towards the treatment area in the treatment state. For example, the outer surface of the porous medium may be positioned radially closer to (e.g. just radially inward of), and/or in contact with, the treatment area, e.g. to enable contact to be established between the porous medium in the treatment area with suction applied to the porous medium. The nasogastric tube may be arranged so that proximal and/or distal ends of the porous medium may expand radially to a greater diameter than a central region of the porous medium.

The nasogastric tube may have: (i) a distal region for insertion into a patient's nasal passageway, (ii) a proximal region for remaining outside the patient, and (iii) a suction region between the proximal and distal regions. The porous medium may be arranged in the suction region. The suction lumen may extend from the proximal region to the suction region (where it is coupled to the porous medium). The nasogastric tube may comprise a drainage lumen extending distally beyond the porous medium for providing drainage of fluid from inside the patient distal to the treatment area. The drainage lumen may extend beyond the suction region to the distal region for providing drainage of fluid from inside the patient distal to the treatment area. The drainage lumen may extend from the proximal region through the suction region and to the distal region. A plurality of fenestrations may be provided in the drainage lumen (e.g. in the distal region of the nasogastric tube). For example, the fenestrations may be provided in a tube which defines the drainage lumen.

The nasogastric tube may be configured to provide both: (i) suction to the porous medium with the porous medium in contact with the treatment area, such as a surgical join, and (ii) drainage of fluid from inside said patient distal to the treatment area using the drainage lumen. This suction and drainage may be provided simultaneously. The treatment area may be located in the patient's GI tract. The treatment area may be located between a patient's nasal passageway and stomach.

The radial deployment system may comprise at least one of: a radial expander arranged to move the porous medium radially outwards to deploy the porous medium from the insertion state to the treatment state; and a removable covering arranged to inhibit movement of the portion of the radial deployment system and/or the portion of the porous medium radially outwards through the window to deploy the porous medium from the insertion state to the treatment state. The removable covering may provide a radial constraint to the porous medium (e.g. to inhibit radial deployment of the porous medium). The porous medium may be configured to expand upon deployment from the insertion state to the treatment state. Expansion of the porous medium may provide the deployment radially outwards (in response to a radial constraint being removed).

The radial expander may comprise a scaffold actuatable to move from a contracted configuration to an expanded configuration to deploy the porous medium from the insertion state into the treatment state. The scaffold may be arranged to be positioned radially within the porous medium and to expand radially outward when moving from the contracted configuration to the expanded configuration. The scaffold may comprise one or more mechanical coupling points arranged to enable a wire to be coupled to that coupling point for controlling actuation of the scaffold by manipulating said wire. The nasogastric tube may comprise one or more said wires, wherein each wire is coupled to a coupling point of the scaffold and is manipulatable from outside the patient to control actuation of the scaffold. The scaffold may comprise a wire construction. The scaffold may be made of a shape memory material biased to return to the expanded configuration. The scaffold may be configured to radially deploy the proximal and/or distal ends of the porous medium to a greater diameter than a central region of the porous medium.

The nasogastric tube may be arranged to provide deployment of the porous medium in response to compression being applied thereto. In particular, the nasogastric tube may be arranged so that applying compression to the porous medium in a longitudinal direction (e.g. a direction substantially parallel to the direction in which the tube extends) causes the porous medium to compress longitudinally and expand radially outwards. For example, the nasogastric tube may be arranged to enable movement of the suction tube (e.g. distally) to cause the compression of the porous medium for deployment. In other words, the radial expander may comprise the arrangement of the suction tube and the porous medium (e.g. so that distal movement of the suction tube causes expansion of the porous medium). The nasogastric tube may be arranged so that a distal end of the porous medium abuts a flange to inhibit further distal movement of the porous medium relative to the rest of the nasogastric tube. Application of a force from the suction tube to the porous medium in a distal direction may cause compression of the porous medium against the flange (thereby providing the corresponding radial expansion of the porous medium). To retract the porous medium (e.g. back into the nasogastric tube from its deployed configuration, the compression may be removed (e.g. by applying a longitudinal force to the porous medium with the suction tube in the opposite direction). This may cause the porous medium to retreat back towards its radially collapsed configuration. The window may extend along a length of the uncompressed porous medium, or it may only extend along a length of the compressed porous medium (e.g. so that deployment of the porous medium radially outwards may be inhibited by the lack of window until sufficient compression of the porous medium is provided).

The porous medium may be arranged so that the amount it expands radially is proportional to the amount it has been compressed longitudinally. The porous medium may be shaped so that under compression its radially outward surface buckles/collapses radially outward. For example, the porous medium may be arranged in a concertina structure and/or the radial expander may comprise a concertina structure arranged radially inward of the porous medium. The concertina structure may be arranged circumferentially so that the outer surface of the porous medium moves radially outward as the concertina structure is compressed. The concertina structure may comprise a series of nodes which are movable relative to other parts of the nasogastric tube (such as the drainage tube) for selective compression of the concertina structure. Porous medium in regions in between the nodes may be configured to bunch and to then move towards a radially expanded treatment position.

The radial expander may comprise a balloon expandable to deploy the porous medium from the insertion state into the treatment state. The balloon may be arranged to be positioned radially within the porous medium and to expand radially outward to deploy the porous medium from the insertion state into the treatment state. The nasogastric tube may comprise a balloon lumen arranged to couple the balloon to a fluid source for controlling deployment of the porous medium. The balloon may be configured to expand so as to radially deploy the proximal and distal ends of the porous medium to a greater radial extent than a central region of the porous medium.

The porous medium may be configured to radially expand to deploy the porous medium into the treatment state. For example, the porous medium may be retained under pressure within a body of the nasogastric tube (e.g. the porous medium may be retained in a compressed state, and biased to return to a radially expanded state). The porous medium may be self-expanding and biased to return to the expanded configuration. The radial expander may comprise a biasing element arranged to bias the porous medium towards the expanded configuration, for example wherein the biasing element is arranged radially within the porous medium. The biasing element may comprise a shape memory material biased to a radially expanded configuration. The porous medium may be configured (e.g. shaped) so that may be longitudinally extended and radially compressed, and vice-versa. The nasogastric tube may be arranged to enable the porous medium to be longitudinally extended (e.g. stretched out) to provide radial compression of the porous medium (for insertion). The nasogastric tube may be arranged to enable the porous medium to be longitudinally shortened to provide radial expansion of the porous medium (for deployment into the treatment state). For example, the nasogastric tube may include an addition component, such as an additional tube, for coupling to the porous medium to selectively control movement between such a longitudinally extended (and radially narrow) state and a longitudinally shortened (and radially extended state). For example, the porous medium may be coil shaped.

A portion of the radial expander may be arranged to move radially outwards through the window to deploy the porous medium from the insertion state into the treatment state. The suction lumen may be coupled to and/or mechanically supported by a portion of the radial expander. The suction lumen may be configured to move with the radial expander as the radial expander moves to deploy the porous medium from the insertion state into the treatment state.

The removable covering may be arranged to exist in: a closed state in which movement through the window to deploy the porous medium from the insertion state to the treatment state is inhibited; and an open state in which movement through the window to deploy the porous medium from the insertion state to the treatment state is enabled. The removable covering may comprise a sheath operable to move relative to the window of the nasogastric tube between a closed position and an open position to provide the closed and open state respectively. The removable covering may comprise a dissolvable member configured to dissolve in response to application of a selected fluid thereto, such as saline.

The nasogastric tube may be operable to provide the window in the suction region through which the porous medium is to be deployed. For example, the nasogastric tube may be configured so that actuation of the nasogastric tube presents the window in the suction region (e.g. to present a window which at least partially radially surrounds the porous medium). A window covering may be provided for the window, which comprises one of: a sheath selectively movable relative to the window between a first position in which the sheath covers the window to inhibit deployment of the porous medium, and a second position in which it enables deployment of the porous medium; and a dissolvable covering configured to dissolve in response to application of a fluid thereto, such as saline. The nasogastric tube may be operable to move the porous medium into the insertion state from the treatment state for removal of the nasogastric tube from the patient. For example, the nasogastric tube may be configured to retract the porous medium so that it is housed within the body carried by the tube, e.g. to facilitate removal of the nasogastric tube and porous medium from the patient.

The nasogastric tube may comprise a lock operable to inhibit reverse deployment of the porous medium from the treatment state into the insertion state. A channel may be provided in the porous medium to enable fluid to flow through the channel of the porous medium between a proximal side of the porous medium and a distal side of the porous medium. The porous medium may be configured to deploy radially outwards through the window about substantially 360 degrees, e.g., wherein the window extends about 360 degrees so that it completely radially surrounds at least some of the porous medium.

The nasogastric tube may comprise a housing arranged to house the components of the nasogastric tube, and wherein the window is provided in the housing. The housing may circumscribe the porous medium, suction lumen, radial deployment system and/or drainage lumen (as well as other components such as a balloon lumen, if present). The housing may be a tube. For example, the housing as a tube may define the outer boundaries of the nasogastric tube. One or more tubes may be provided within the housing. For example, a drainage tube may be located in the housing. The drainage tube may define the drainage lumen (e.g. the drainage lumen may comprise the region circumscribed by the drainage tube). The region radially inward of the housing (e.g. and radially outward of the drainage tube, if present) may provide the suction lumen. In other words, the housing may be a tube which circumscribes the suction lumen (e.g. to define the suction lumen). Where a drainage lumen is provided (and this is defined by the drainage tube), the nasogastric tube may be a two-tube device (e.g. with one tube, the drainage tube, inside of another tube, the housing). The suction lumen may be defined by an additional and/or alternative component to the housing. For example, a suction tube may be provided which defines the suction lumen. The suction tube may be contained within the housing. The suction tube may be provided in addition to the drainage tube, e.g. to provide a device with two tubes within a tube (a three-tube device). The different tubes may each move relative to one another.

The housing may define an internal housing lumen which runs from a proximal location (e.g. outside a patient) to a distal region (e.g. inside the patient). The other components of the nasogastric tube (e.g. porous medium, suction lumen, radial deployment system, drainage lumen) may be arranged within the suction lumen. In examples, the internal housing lumen may provide one of the other lumens, such as the suction lumen. Alternatively, the internal housing lumen may provide a housing in which other components are provided. For example, the drainage lumen and the suction lumen may be tubes which run within the housing (e.g. in the housing lumen). In other words, the drainage lumen may be provided by a tube (drainage tube) and the suction lumen may be provided by a separate tube (suction tube). Those two tubes may be provided inside another tube (e.g. the housing tube), e.g. so that the drainage and suction tubes run inside the housing tube. The porous medium may be located internally in the insertion state and externally in the treatment state. For example, the porous medium may be located within the housing in the insertion state. At least some of the porous medium may be located outside the housing in the treatment state. A surface of the housing is selectively movable to either cover or uncover the opening through which the porous medium will pass. The nasogastric tube (e.g. a body of the nasogastric tube, such as the housing) may be arranged to retain the porous medium in a compressed state for insertion into said patient's body. The tube may be arranged to enable expansion as part of, or for, deployment of the porous medium towards the treatment area.

The nasogastric tube may comprise a lumen coupling at the proximal region. The lumen coupling may be configured to couple: (i) the suction lumen to a negative pressure source, and (ii) the drainage lumen to a drainage component. The lumen coupling may provide an active coupling for the suction lumen, so that the suction lumen is coupled to a source of negative pressure arranged to actively apply suction to the suction lumen (and thus also the porous medium/treatment area). The lumen coupling may provide a separate coupling for coupling the drainage lumen to the drainage component. The lumen coupling to the drainage component may be arranged to work with multiple different drainage components, e.g. so that the drainage lumen may be coupled to one or more of a plurality of different drainage components. The lumen coupling may comprise a single component arranged to enable different components to be coupled to the drainage and suction lumens. For example, the lumen coupling may be configured to couple the drainage lumen to a receptacle for receiving fluid from the patient distal to the treatment area (e.g. a drainage bag). For example, the lumen coupling may be configured to couple the drainage lumen to a syringe, e.g. the lumen coupling may comprise a syringe coupling which couples the drainage lumen to a syringe. The lumen coupling may provide an active coupling for the suction lumen (e.g. by coupling the suction lumen to an active source of negative pressure). The lumen coupling may provide a passive and/or active coupling for the drainage lumen (e.g. by coupling the drainage lumen to a passive component such as a receptacle, or an active component such as a syringe). The tube may be configured to enable active/passive components to be used interchangeably with the drainage lumen (e.g. so that active drainage with a syringe and passive drainage into a drainage bag may be used sequentially). The lumen coupling may be configured to also couple the balloon lumen to a source of fluid for selectively controlling expansion of the balloon.

### FIGURES

Some examples of the present disclosure will now be described, by way of example only, with reference to the figures, in which:

FIGS. 1a to 1d show schematic illustrations of an exemplary method of using a nasogastric tube for treatment of an internal surface of a patient.

FIGS. 2a and 2b are schematic diagrams of an exemplary nasogastric tube.

FIGS. 3a and 3b are schematic diagrams of an exemplary nasogastric tube.

FIGS. 4a and 4b are schematic diagrams of an exemplary nasogastric tube.

In the drawings like reference numerals are used to indicate like elements.

### Specific Description

Embodiments of the present disclosure are directed to a nasogastric tube for treating a treatment area internal to a patient. The nasogastric tube may include a porous medium and a radial deployment system. The tube may be inserted through a patient's nasal passageway into their GI tract, and then located so that it lies radially inwards from an internal surface of a body lumen of the GI tract. The tube may then be positioned with the porous medium aligned with a treatment area on the internal surface of the body lumen of their GI tract, such as a surgical join (e.g. from an oesophagectomy). Once in this position, the radial deployment system may be actuated to deploy the porous medium radially outwards so that an outer surface of the porous medium comes into contact with the treatment area on the internal surface of the body lumen, or is at least arranged just radially inwards of the treatment area (e.g. so that application of suction to the porous medium will bring the treatment area into contact with the porous medium). Suction may then be applied to the porous medium (and thus to the treatment area) using a suction lumen of the nasogastric tube. For example, where the porous medium is deployed so that it is close to, but not fully in contact with, the treatment area, the application of suction to the porous medium may bring the treatment area into contact with the porous medium. The nasogastric tube may also include a drainage lumen which extends beyond the porous medium and further into the patient's GI tract. The drainage lumen may provide drainage of fluids further within the patient's GI tract.

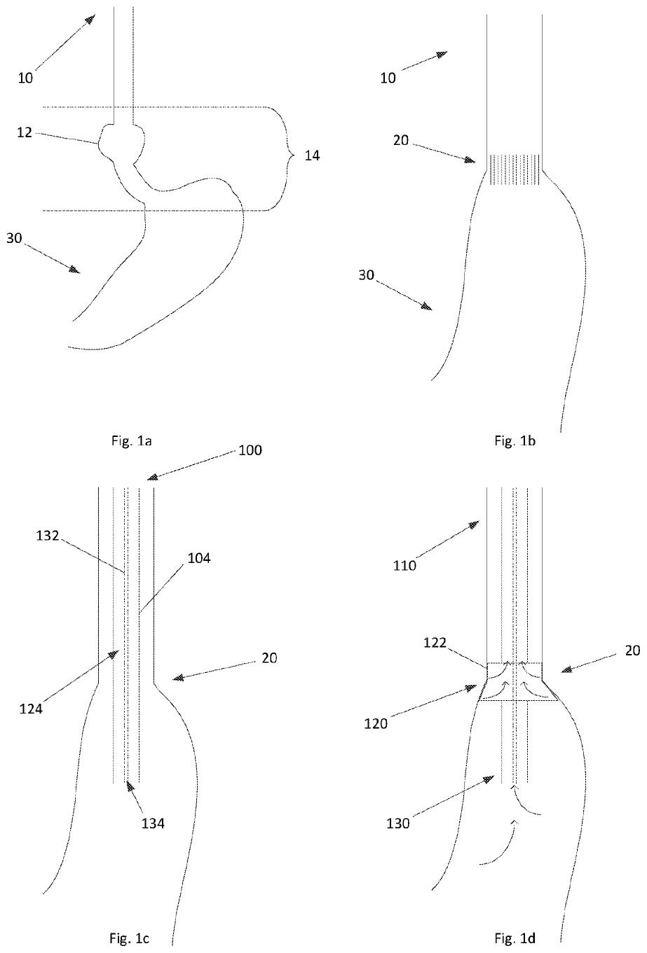

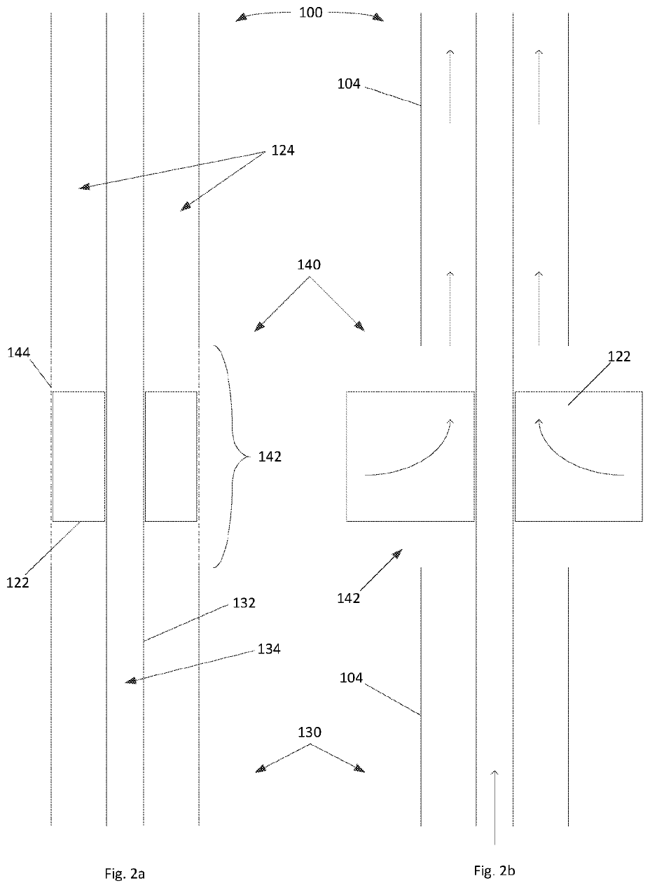

A method of using such a nasogastric tube will now be described with reference to FIGS. 1a to 1d, then exemplary nasogastric tubes will be described with reference to FIGS. 2 to 4.

The following description relates to use of such a nasogastric tube after an oesophagectomy, but is to be appreciated that this should not be considered limiting, and that the nasogastric tubes of the present disclosure may be used in connection with other medical procedures.

FIG. 1a shows a portion of a patient's GI tract. For simplicity, only a small portion of the GI tract is shown, but it will be appreciated that the GI tract provides a continuous body lumen extending from the mouth to anus. FIG. 1a shows an upper portion 10 of the GI tract through to a lower portion 30 of the GI tract, which in the example shown in FIG. 1a comprises the patient's stomach although the distal portion could be located further into the GI tract (e.g. in the patient's bowels). Located between the upper portion 10 and the lower portion 30 is a tumour 12. Typically, such a tumour 12 will be provided in the bottom third of the oesophagus, near to the top of the stomach. In which case, the tumour 12 and surrounding tissue (lower part of the oesophagus and upper part of the stomach) may be removed. The portion of the patient's oesophagus proximal to the removed area is then surgically joined to the portion of the patient's stomach distal to the removed area. In other cases (where the tumour is higher in the patient's oesophagus), a portion of the patient's bowel may also be removed and used to connect their oesophagus to their stomach after the tumour and surrounding region of the oesophagus has been removed.

In the example shown in FIG. 1a, the tumour 12 is located in the patient's oesophagus near to their stomach. An area to be removed 14 is shown in dashed lines in FIG. 1a. The area to be removed 14 includes the tumour, a distal end of the oesophagus, and a proximal end of the stomach. Once this area 14 has been removed, the remaining portions of the patient's oesophagus and stomach are re-joined. This is shown in FIG. 1b. FIG. 1b provides a zoomed in view relative to that of FIG. 1a. As can be seen in FIG. 1b, a treatment area 20 is shown by a series of lines. The treatment area 20 is on an internal surface of the internal body lumen (e.g. an internal surface of the GI tract lumen). The treatment area 20 may comprise a pre-defined area of weakness/vulnerability, such as a surgical join. In the example shown, the treatment area 20 comprises a region of the GI tract where two separate parts have been surgically joined together (in this example, the oesophagus and the stomach). The treatment area 20 may be an area deemed susceptible to a defect forming, or it may be an area which may not be capable of withstanding substantial stress or pressure being applied thereto. In the case of an oesophagectomy, the treatment area 20 comprises a surgical join which could rupture leading to a leakage of bowel contents into tissues surrounding the join, and thus giving rise to subsequent abscess formation and sepsis. Nasogastric tubes of the present disclosure are configured to facilitate treatment without such rupturing occurring.

FIGS. 1c and 1d show a nasogastric tube 100 of the present disclosure inserted into the patient. In FIG. 1c, the nasogastric tube 100 is in an insertion state, and in FIG. 1d, the nasogastric tube is in a treatment state. The nasogastric tube 100 defines two lumens: a suction lumen 124 and a drainage lumen 134. A housing 104 of the nasogastric tube 100 defines the suction lumen 124. A drainage tube 132 of the nasogastric tube 100 defines the drainage lumen 134. The drainage tube 132/drainage lumen 134 is located radially within the housing 104. The drainage tube 132/drainage lumen 134 is shown in dashed lines. As can be seen in FIG. 1d, the nasogastric tube 100 has a proximal region 110, a suction region 120, and a distal region 130. The suction region 120 is arranged between the proximal and distal regions of the tube 100.

The nasogastric tube 100 is inserted through the patient's nasal passageway and into their GI tract. The nasogastric tube 100 is inserted so that the distal region 130 of the tube 100 extends down to the lower portion of the GI tract, which in this example is into the patient's stomach. A portion of the proximal region 110 of the tube 100 will remain outside the patient. Features of the nasogastric tube 100 in the proximal region 110 outside the patient may be controlled/manipulated by a doctor in a region outside the patient to control operation of the nasogastric tube 100 inside the patient. The tube 100 is inserted into the patient so that the suction region 120 is located radially inward of the treatment area 20. For example, the suction region 120 of the nasogastric tube 100 may be aligned with the treatment area 20 (e.g. so that the two are adjacent), with the suction region radially within the treatment area 20 in the body lumen of the patient.

Between FIG. 1c and FIG. 1d, the nasogastric tube 100 is actuated to change from its insertion state (FIG. 1c) to its treatment state (FIG. 1d). FIG. 1d shows the nasogastric tube 100 in its treatment state and being used to provide treatment to the patient. The nasogastric tube 100 includes a porous medium 122 which is arranged in the suction region 120 of the tube 100. The porous medium 122 is aligned with the treatment area 20 so that it may be deployed radially outwards to bring an outer surface of the porous medium 122 into contact with, or just radially inward of, the treatment area 20 on the inner surface of the body lumen. With the porous medium 122 in this position, suction is then applied to the porous medium 122 (this is shown by the arrows in FIG. 1c). In turn, this causes suction to be applied to bring the treatment area 20 of the inner surface of the body lumen into contact with the porous medium 122 (if the outer surface of the porous medium 122 was not already in contact with the treatment area 20).

Applying suction to the porous medium 122 with the outer surface of the porous medium 122 in contact with the treatment area may provide treatment to the treatment area 20. For example, this may inhibit damage occurring to the treatment area 20, and/or facilitate healing of that area. Applying suction to the porous medium 122 when in contact with the treatment area 20 may also provide control to the treatment area. For example, if a hole were to form in the treatment area 20 (e.g. if the surgical join were to deform in some way), then the application of suction to that region and the presence of the porous medium 122 in that region could inhibit any deleterious effects from occurring. In particular, this may inhibit bowel contents from leaking outwards through that hole (which would give rise to other issues such as abscess formation/sepsis etc.).

The duration for which this suction is applied may vary depending on the particular issue to be treated, but typically, the porous medium 122 may remain in place providing suction to the treatment area 20 of the patient for up to a week. After the nasogastric tube 100 is removed, the patient will be examined for the presence of any defects. If a defect was still present (e.g. in the region of the treatment area 20), then a new nasogastric tube 100 may be inserted (and porous medium 122 deployed) for a subsequent treatment period. This process would be repeated until the defect is healed. While the patient is being treated with the porous medium 122, they may be given essential nutrients/fluids etc. These may be provided intravenously, or enterally such as via a surgical jejunostomy.

The nasogastric tube 100 may also be used to provide drainage from the lower portion of the patient's GI tract. Excess fluids may build up in the patient's GI tract, and these may be drained using the nasogastric tube 100. The tube 100 may be operated to provide active and/or passive draining. For example, a syringe could be coupled to the tube 100 in the proximal region 110 to enable actuation of that syringe to provide active drainage. Additionally, and/or alternatively, a receptacle may be coupled to the tube 100 to receive fluids which were drained passively from the tube 100. The arrows shown in FIG. 1d indicate passage of fluids in the lower portion of the GI tract into the tube 100 for drainage. In particular, these drainage fluids will pass into the drainage lumen 134 of the nasogastric tube 100. The drainage may occur simultaneously with suction being applied to the porous medium 122. The arrows in FIG. 1d also show suction being applied to the porous medium 122. The suction is applied via the suction lumen 124. The drainage lumen 134 is separated and isolated from the suction lumen so that fluid and/or suction does not pass between the two lumens.

An example of a nasogastric tube 100 will now be described with reference to FIGS. 2a and 2b.

FIGS. 2a and 2b show cross-section views of a nasogastric tube 100 including a porous medium 122. In FIG. 2a, the porous medium 122 is in an insertion state and in FIG. 2b, the porous medium 122 has been deployed into a treatment state. In FIG. 2b, the line arrows indicate flow of fluid.

The nasogastric tube 100 includes a first radial deployment system 140. The radial deployment system 140 includes a first window 142 and a covering 144.

The nasogastric tube 100 defines a suction lumen 124 and a drainage lumen 134. The nasogastric tube 100 includes a housing 104. The housing 104 defines the suction lumen 124. The nasogastric tube 100 includes a drainage tube 132. The drainage tube 132 defines the drainage lumen 134. The drainage tube 132 is located radially inward of the housing 104. The suction lumen 124 is provided by the region circumscribed by the housing 104 and radially outward of the drainage tube 132. In this example, the suction lumen 124 is annular. The drainage lumen 134 is provided by the region circumscribed by the drainage tube 132. In this example, the drainage lumen is cylindrical.

A distal region 130 of the nasogastric tube 100 is shown in FIGS. 2a and 2b. Although not shown, the top of the nasogastric tube 100 in FIGS. 2a and 2b extends in a proximal direction to a proximal region 110 of the tube 100. The porous medium 122 and first radial deployment system 140 are arranged in a suction region 120 of the nasogastric tube 100. The nasogastric tube 100 extends beyond the suction region 120 to the distal region 130, such that the two regions are longitudinally offset from each other. In the example shown in FIGS. 2a and 2b, both the drainage tube 132 and the housing 104 extend distally from the suction region 120.

The nasogastric tube 100 extends from its proximal region 110 through to its distal region 130. The suction region 120 is located between the proximal and distal regions. FIGS. 2a and 2b show cross-section views in a plane running through a longitudinal axis of the tube 100. The nasogastric tube 100 may be cylindrical along its length (e.g. the cross-sectional plane may be along a diameter of the cylinder).

The suction lumen 124 extends from the proximal region 110 to the suction region 120, where it is coupled to the porous medium 122. In the example shown in FIGS. 2a and 2b, the suction lumen 124 is annular. The drainage lumen 134 extends from the proximal region 110 to the distal region 130 (through the suction region 120). The drainage lumen 134 is located centrally (e.g. along a longitudinal axis of the tube 100). The drainage lumen 134 is sealed by the drainage tube 132. The drainage lumen 134 is separate to, and isolated from, the suction lumen 124 and porous medium 122 (e.g. so that the drainage lumen 134 is not in fluid communication with either the porous medium 122 or the suction lumen 124). The suction lumen 124 is located radially outward from the drainage lumen 134. For example, the drainage tube 132 and the housing 104 may be coaxial. In this example, the suction lumen 124 is annular and so circumscribes the drainage lumen 134. The porous medium 122 is located radially outward of the drainage lumen 134/drainage tube 132.

The housing 104 extends from the proximal region 110 to the distal region 130. The housing may carry a body of the nasogastric tube 100, or the housing itself may provide a body of the nasogastric tube 100. For example, the housing 104 may define an outer surface of the nasogastric tube 100 (e.g. while the porous medium 122 is in the insertion state). The housing circumscribes the suction lumen 124 and the drainage tube 132/drainage lumen 134. The housing 104 also circumscribes the porous medium 122 when the porous medium 122 is in the insertion state.

The first window 142 is defined in the housing 104 in the suction region 120. The first window circumscribes the porous medium 122, e.g. the first window 142 is located radially outward from the porous medium 122. With the porous medium 122 not deployed, and the tube 100 in an insertion state (as shown in FIG. 2a), the covering 144 obstructs the first window 142. The covering 144 circumscribes, or fills, the first window 142, and thus also circumscribes the porous medium 122. The covering 144 is shown by dashed lines in FIG. 2a. The first window may comprise an opening, such as an aperture, in a surface of a body of the nasogastric tube 100. For example, the first window 142 may comprise an aperture in a tangential surface of the body (e.g. housing 104).

The nasogastric tube 100 is configured to be inserted into a patient's nasal passageway. The tube 100 is configured to be moved through the nasal passageway and into the GI tract to place the tube 100 in a desired location in the patient's GI tract. Typically, the desired placement of the nasogastric tube 100 will include placement of the distal region 130 of the tube 100 in a region of the patient's body where drainage may be useful (e.g. their stomach or bowel). The desired placement of the tube 100 may include placement of the suction region of the tube 100 in a body lumen inside the patient's body (e.g. their GI tract) where an internal surface of that body lumen has a treatment area 20.

The tube 100 is arranged to be positioned with the suction region 120 (porous medium 122, first window 142, and covering 144) adjacent to the treatment area 20. In other words, the porous medium 122, first window 142 and covering 144 may be aligned with the treatment area 20 and/or located radially inward of the treatment area 20.

The first radial deployment system 140 is configured to selectively deploy the porous medium from an insertion state into a treatment state. In the treatment state, the porous medium may be arranged with an outer surface of the porous medium 122 in contact with, or just radially inward of, the treatment area 20. In the insertion state, the outer surface of the porous medium 122 may be arranged radially inwards as compared to when in the treatment state. In other words, the footprint of the tube 100 may be smaller (e.g. when viewed in plan, the area/width of the tube 100 may be smaller). The first deployment system 140 may be configured to control movement of the outer surface of the porous medium 122 from a narrower position (in the insertion state) into a wider position (in the treatment state). The first deployment system 140 may be configured to deploy the porous medium 122 so that it may be used to provide treatment to the treatment area 20 (e.g. after it has been aligned radially within the treatment area 20).

The first radial deployment system 140 is configured to inhibit radially outward movement until it is controlled to deploy the porous medium 122. As can be seen in FIG. 2a, the porous medium is retained in a narrower configuration (e.g. in a compressed state) by the covering 144. As can be seen in FIG. 2b, the porous medium 122 is in a wider configuration (e.g. an expanded state), as the covering 144 has been removed.

The porous medium 122 is arranged to be biased radially outwards. For example, the porous medium 122 may be folded and/or compressed so that it is small enough to be inserted into the tube 100, e.g. within the housing 104. The covering 144 may be arranged to provide a radial restraint to the radially outward biasing of the porous medium 122. The covering 144 may be arranged to inhibit radial expansion of the porous medium 122 through the first window 142. The covering 144 may be configured to withstand stress exerted by the porous medium attempting to expand radially outward. The covering 144 may retain the porous medium in its insertion state (e.g. biased to expand but unable to do so as the covering 144 is restraining this movement). In other words, the covering 144 may be arranged to selectively close the first window 142. The covering 144 may block movement out through the first window 142.

The first radial deployment system 140 is configured to control deployment of the porous medium 122 from the insertion state into the treatment state by restraining the porous medium from expanding until expansion is desired. For example, to deploy the porous medium 122, the device may be actuated to remove this radial restraint on the porous medium 122. Removing the radial restraint may comprise removing the covering 144 from obstructing the first window 142. In other words, the first window 142 may be opened by removal of the covering 144 so that the porous medium 122 may expand radially outwards through the window 142 into the treatment state (e.g. so that the outer surface of the porous medium 122 may be positioned in contact with, or just radially inward of, the treatment area 20). The porous medium 122 may be aligned with the window 142 to enable deployment of the porous medium radially outwards and through the window 142 (once the covering 144 has been removed).

The covering 144 may be configured to be dissolvable in response to application of a selected fluid thereto. The selected fluid may comprise a fluid compatible with insertion into a patient's GI tract. For example, the covering 144 may be configured to dissolve in the presence of saline. The nasogastric tube 100 may be configured to deliver saline to the covering 144 to remove said covering 144. Saline may be flushed through the tube 100 so that it comes into contact with the covering 144. For example, saline may be delivered into the suction lumen 124. The suction lumen 124 is coupled to the porous medium 122, and so the saline may be delivered to the porous medium 122 through the suction lumen 124. The porous medium 122 is in contact with the covering 144, and so saline may pass through the porous medium 122 and come into contact with the covering 144. As the covering 144 dissolves, the radially constraint on the porous medium 122 may be removed to enable the porous medium 122 to self-expand radially outward through the first window 142.

The suction lumen 124 is coupled to the porous medium 122 and configured to selectively provide suction thereto. The suction lumen 124 may be arranged to provide suction to the porous medium 122 when in the treatment position. Suction may thus be applied to the porous medium 122, and to the outer surface of the porous medium 122, to provide suction to the treatment area 20. The nasogastric tube 100 is arranged so that, with the porous medium 122 deployed in the treatment state and suction being applied to the porous medium 122, a portion of the treatment area 20 will be in contact with an outer surface of the porous medium 122. This may provide a healing effect to the treatment area 20.

The drainage lumen 134 may be configured to provide active and/or passive drainage of fluids from the GI tract. The drainage tube 132 is solid and separates the drainage lumen 134 from the suction lumen 124 along the length of the suction lumen 124 (e.g. so that the two lumens are not in fluid communication with each other). The drainage tube 132/drainage lumen 134 is arranged to be positioned further into the GI tract than the porous medium 122. For example, the porous medium 122 may be positioned somewhere between mouth and stomach (e.g. at a surgical join from an oesophagectomy), whereas the drainage lumen 134 may extend to the distal region 130 of the nasogastric tube 100, e.g. to be positioned further into the patient, such as in their stomach or bowels. A proximal end of the drainage lumen 134 may be coupled to an active drainage source, such as a syringe, to provide active draining from within the patient using the drainage lumen 134. Alternatively, and/or additionally, the drainage lumen may be coupled to a receptacle arranged to receive fluids which were passively drained through the drainage lumen 134.

The housing 104 may be arranged to carry, or provide, a body for the nasogastric tube 100. For example, the housing 104 may provide an outer wall within which all of the other components of the nasogastric tube 100 are placed (except from the porous medium 122 when in the treatment state). The first window 142 may extend about 360 degrees. For example, a region of the housing 104 proximal to the first window 142 may be coupled to a region of the housing 104 distal to the first window 142 radially inwards of the porous medium 122. In other words, the housing 104 may be arranged to enable 360 degrees of radially outward deployment for the porous medium 122. The housing 104 may be coupled to the drainage lumen 134. For example, the housing 104 may be coupled to the drainage lumen either side of the first window 142. The housing 104 may define a recess in the tube 100 in which the porous medium 122 may be provided so that the porous medium 122 may be deployed radially outwards through the first window 142 without obstruction from the wall of the housing 104 (once the covering 144 has been removed).

The porous medium 122 may be sized so that it is compressible to fit within the housing 104. The porous medium 122 comprises a material having pores which are typically of a size between 400 to 600 microns. The porous medium 122 may include one or more materials such as: (i) foams e.g. a polyurethane foam, (ii) expandable meshes e.g. a wire mesh, (iii) bio-active materials e.g. bio-active collagen, (iv) an open-pore drainage film. For example, the wire mesh may be formed of a shape memory material, such as a nickel titanium alloy (e.g. nitinol). The porous medium 122 may be configured to be located internally when in the insertion state. For example, the porous medium 122 may be configured so that it remains radially inside the housing 104/covering 144 when in the insertion state. The porous medium may be configured to be located externally when in the treatment state. For example, the porous medium 122 may be configured to self-expand in response to removal of the covering 144, so that it extends radially outward from the housing 104 when in the treatment state.

In operation, the nasogastric tube 100 may be inserted into the patient's nasal passageway and positioned inside the patient, as described above with reference to FIGS. 1a to 1d. The nasogastric tube 100 will be positioned so that the porous medium 122 lies radially within the window 142 and covering 144, which are radially within the treatment area 20. In other words, the nasogastric tube 100 is positioned so that the porous medium 122 is aligned with the window 142 so that it may be deployed radially outwards through the window 142 and into contact with, or just radially inward of, the treatment area 20.

Once the nasogastric tube 100 is in position, saline (or another suitable dissolving fluid) is flushed through the suction lumen 124. The saline passes into the porous medium 122 and onto the covering 144, which in turn dissolves. The covering 144 is therefore dissolved, leaving open the first window 142. As the porous medium 122 is biased to expand radially outwards, and its radial constraint has been removed, the porous medium 122 will itself expand causing its outer surface to move radially outward through the first window 142. The outside surface of the porous medium 122 will thus be moved into contact with, or radially closer, the treatment area 20. Suction is then applied to the porous medium 122 through the suction lumen 124 which will cause the treatment area 20 to remain in contact with the porous medium 122 for treatment. Simultaneously to this happening, the drainage lumen 134 may provide active or passive drainage of fluids further inside the patient's GI tract than the treatment area 20. Once sufficient treatment has been provided (e.g. after 1 to 5 days), the nasogastric tube 100 may be removed with the porous medium 122 in the treatment state. Alternatively, the porous medium 122 may be pulled radially inwards by pulling proximally on a component of the nasogastric tube such as a retraction mechanism (e.g. which is coupled to the porous medium to facilitate retraction of the porous medium from the treatment state into the insertion state for removal from the patient).

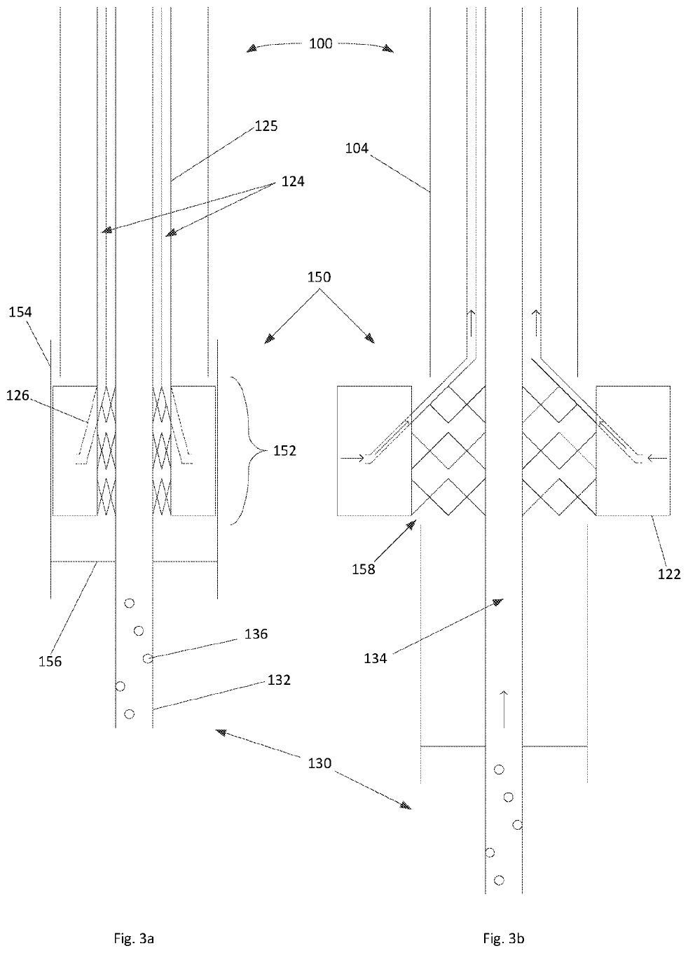

Another example of a nasogastric tube 100 will now be described with reference to FIGS. 3a and 3b. Some of the features of this exemplary nasogastric tube 100 may be similar to those described above with reference to FIGS. 2a and 2b, and so these features shall not be described again. In FIG. 3b, the line arrows indicate flow of fluid.

FIGS. 3a and 3b show cross-section views of a nasogastric tube 100 including a porous medium 122. In FIG. 2a, the porous medium 122 is in an insertion state and in FIG. 2b, the porous medium 122 has been deployed into a treatment state.

The nasogastric tube 100 includes a second radial deployment system 150. The radial deployment system 150 includes a second window 152 and a scaffold 158. The second radial deployment system 150 may also include a sheath 154 and a sheath coupling 156.

The nasogastric tube 100 includes a suction lumen 124 and a drainage lumen 134. As with the example shown in FIGS. 2a and 2b, the nasogastric tube 100 includes a drainage tube 132 which defines the drainage lumen. A plurality of fenestrations 136 are provided in the drainage tube 132 (at its distal end) to provide multiple access points to the drainage lumen 134 in that region. The suction lumen 124 has a distal end coupling 126. The nasogastric tube 100 also includes a housing 104. Unlike in the example shown in FIGS. 2a and 2b, the housing 104 does not define the suction lumen 124. Instead, the nasogastric tube 100 includes a suction tube 125, which defines the suction lumen 124. The suction tube 125 is provided radially within the housing 104. The drainage tube 132 is also provided radially within the housing 104. Thus, in this example, two separate tubes (inner and suction) are provided inside an outer tube (housing). The suction tube 125 may be annular (as shown in FIGS. 3a and 3b), or any other suitable shape, such as cylindrical.

As with FIGS. 2a and 2b, a distal region 130 of the tube 100 is shown in FIGS. 3a and 3b. Although not shown, the top of the tube 100 in FIGS. 3a and 3b extends in a proximal direction to a proximal region 110 of the tube 100. The porous medium 122 and second radial deployment system 150 are arranged in a suction region 120 of the tube 100. The tube 100 extends beyond the suction region 120 to the distal region 130, such that the two regions are longitudinally offset from each other.

The scaffold 158 is arranged radially within the porous medium 122. The suction lumen 124 is coupled to the porous medium 122. As shown, the distal end coupling 126 of the suction lumen 124 may also be coupled to the scaffold 158. The porous medium 122 is aligned with the second window 152 in the suction region 120 of the tube 100. As shown in FIG. 3a, the sheath 154 is located radially outward from the porous medium 122 and the scaffold 158. In the insertion state (FIG. 3a), the sheath 154 is positioned to cover the second window 152. In the treatment state (FIG. 3b), the sheath 154 is positioned to not cover the second window 152. The sheath 154 may at least partially overlie a portion of the housing 104 (e.g. in the suction region 120). In other words, a proximal portion of the sheath 154 may at least partially circumscribe a portion of the housing 104. However, it is to be appreciated that the sheath 154 could lie radially within the housing 104, and/or the sheath 154 could abut the housing 104, with the two being located at the same distance radially outward. As shown in FIG. 3a, the outer surface of the porous medium 122 may be arranged radially outward of the housing 104. In other words, a portion of the porous medium 122 may be arranged outside the window 152 in the insertion state (e.g. but retained radially within the sheath 154). The scaffold 158 is coupled to the porous medium 122 (e.g. the two may be attached) so that they move together. In other words, the scaffold 158 may be coupled to the porous medium 122 so that radial expansion of the scaffold 158 (e.g. in response to inflation) may cause the porous medium to also move radially outwards, and vice-versa.

The sheath 154 may be coupled to the drainage tube 132. The sheath 154 may be coupled to the drainage tube 132 via the sheath coupling 156. As shown in FIGS. 3a and 3b, a distal portion of the sheath 154 is coupled to the drainage tube 132 drainage lumen 134 via the sheath coupling 156. The sheath coupling 156 may extend radially outward from the drainage tube 132 to the sheath 154. As with the other FIGS. the drainage lumen 134 extends to the distal portion of the tube 100. The drainage tube 132 has a plurality of fenestrations 136 in its distal region 130. The fenestrations 136 provide fluid flow paths into the drainage lumen 134 (e.g. from regions in addition to the hole at the end of the drainage lumen 134).

The scaffold 158 is arranged to provide radially outward movement of the porous medium 122. In the insertion state, the scaffold 158 is arranged in a narrower (contracted) configuration. The porous medium 122 is arranged radially outward of the scaffold 158 but radially inward of the sheath 154 in the narrower configuration. In the treatment state, the scaffold 158 is arranged in a wider (expanded) configuration. The porous medium 122 is radially outward of the scaffold 158. The porous medium 122 is radially outward of the second window 152 and the sheath 154 for treatment of the treatment area 20. The suction tube 125/suction lumen is coupled to the scaffold 158 to move with the scaffold 158 when the scaffold 158 expands from the insertion state to the treatment state. For example, the scaffold 158 may comprise a number of elements (e.g. wires) which make up the scaffold 158, and the distal end coupling may be coupled to one of said elements. The suction tube 125/suction lumen 124 (and distal end coupling 126) may move with the scaffold 158 so that they are coupled to the porous medium 122 with the porous medium 122 in the treatment state. The scaffold 158 may be coupled to the drainage tube 132 (e.g. a radially inner portion of the scaffold 158 may couple to a radially outward surface of the drainage tube 132). It is to be appreciated in the context of the present disclosure that the suction lumen may be of the type shown in FIGS. 2a and 2b. For example, the porous medium may be arranged at a distal end of the suction lumen (when in the insertion state). The suction lumen may not move with the porous medium as the porous medium is deployed radially outward, but it may still provide suction to the porous medium in its deployed configuration.

The sheath 154 may be arranged to move relative to the second window 152. The sheath 154 may also be arranged to move relative to the housing 104. For example, the sheath 154 may be arranged to move from a closed position (in which deployment of the porous medium 122 is inhibited-FIG. 3a) to an open position (in which deployment of the porous medium 122 is enable-FIG. 3b). Movement of the sheath 154 may comprise sliding relative to the housing 104, e.g. sliding in a proximal (e.g. up the GI tract) or distal (down the GI tract) direction. The sheath 154 may be arranged to slide over the housing 104. The sheath coupling 156 may couple the sheath 154 to the drainage tube 132 so that movement of the drainage tube 132 provides movement of the sheath 154. For example, the tube 100 may be configured so that proximal/distal movement of the drainage tube 132 provides a corresponding proximal/distal movement of the sheath 154. In other words, the drainage tube 132 may be arranged to actuate movement of the sheath 154 between the closed position and the open position. As can be seen in FIG. 3b, the drainage tube 132/drainage lumen 134 has been moved distally, thereby causing a corresponding distal movement of the sheath 154 to open the second window 152.

It is to be appreciated in the context of the present disclosure that features of different radial deployment systems may be combined, and/or that not all features of the radial deployment systems shown in the FIGS. are needed. Radial deployment systems of the present disclosure may comprise a radial expander arranged to move the porous medium 122 radially outwards to deploy the porous medium 122 from the insertion state to the treatment state, and/or a removable covering arranged to inhibit movement of the portion of the radial deployment system and/or the portion of the porous medium 122 radially outwards through the window to deploy the porous medium 122 from the insertion state to the treatment state.

For the second radial deployment system 150 shown in FIGS. 3a and 3b, the scaffold 158 may provide the radial expander, and the sheath 154 may provide the removable covering. That is, the scaffold 158 is operable to provide radial deployment of the porous medium 122 and the sheath 154 is operable to inhibit that radial deployment.

To provide radial deployment of the porous medium 122, the scaffold 158 is arranged to expand radially outward, thereby to provide movement of the porous medium 122 radially outward (e.g. to move the outer surface of the porous medium 122 into location for treatment). The scaffold 158 may be self-biased to expand radially outwards, and/or the scaffold 158 may be actuatable to expand radially outwards.

For a self-expanding scaffold 158 (e.g. a scaffold self-biased to return to its expanded configuration), the scaffold 158 may be made of a shape memory material. For example, the scaffold 158 may comprise a Nickel-Titanium alloy, such as Nitinol. The scaffold 158 may be arranged to have an expanded configuration towards which the scaffold 158 is biased. As such, in response to compressing the scaffold 158, the scaffold 158 will remain biased to return to that expanded configuration. In such examples, the scaffold 158 may provide a biasing element arranged to bias the porous medium 122 towards the treatment state. For example, the scaffold 158 may be arranged radially within the porous medium 122, and its expanded state may include a radial expansion of the scaffold 158, so that when the scaffold returns to its expanded state, it moves the porous medium 122 into the treatment position. The self-biased scaffold 158 may be provided in combination with a removable covering for the window 152. For example, a dissolvable covering may be provided (as shown in FIGS. 2a and 2b), and/or a movable sheath 154 may be provided (as shown in FIGS. 3a and 3b).

Where the scaffold 158 of FIGS. 3a and 3b comprises a self-expanding scaffold 158, the movable sheath 154 may be arranged to inhibit radial expansion of the scaffold 158 to its expanded state when in the closed position. In other words, the sheath 154 may be arranged to block the second window 152 to inhibit radial expansion of the scaffold 158 and movement out through the window 152. The sheath 154 may be arranged so that when in the open position, the window 152 is no longer obstructed, and so the scaffold 158 may expand radially outwards towards its expanded configuration to deploy the porous medium 122.

For an actuatable scaffold 158, the scaffold 158 may be coupled to an additional component for controlling actuation of the scaffold 158. Controlling actuation of the scaffold 158 may comprise controlling selective deployment of the scaffold 158 (e.g. selectively activating from insertion state to treatment state). Controlling actuation of the scaffold 158 may comprise controlling the extent of radial deployment of the scaffold 158 (e.g. selecting a radial expansion diameter for the scaffold 158, such as to fit the deployed porous medium to the size of the body lumen). For example, an actuator may be provided in the proximal region 110 of the tube (outside the patient), which is coupled to the scaffold 158. Control of the actuator (outside the body) may selectively actuate the scaffold 158. In other words, the actuator may be controlled to selectively move the scaffold 158 between its contracted configuration and its expanded configuration (as well as to select the extent of radial expansion for the scaffold 158). For example, the actuator may be coupled by one or more coupling wires to the scaffold 158, so that those coupling wires may be controlled to provide expansion/collapsing of the scaffold 158. This is not shown in FIGS. 3a and 3b, but one or more coupling wires may extend from the scaffold 158 in suction region 120 of the tube 100 to the proximal region 110 of the tube 100 (e.g. where they are coupled to the actuator).

The scaffold 158 may comprise a construction of individual elements, such as wires, which are arranged together to provide an expandable framework. In particular, the scaffold 158 may comprise one or more mechanical coupling points for coupling the scaffold 158 to an actuator. The mechanical coupling points may comprise a coupling for a coupling wire. The scaffold 158 may be arranged so that increasing/decreasing tension of each coupling wire coupled to a coupling point of the scaffold 158 may cause movement of one or more of the elements of the scaffold 158. For example, the elements of the scaffold 158 coupling to a particular coupling point may move towards the point in response to increased tension in the coupling wire coupled to that coupling point. The scaffold 158 may be arranged so that manipulation of the actuator/coupling wires in this manner may cause a movement of the elements of the scaffold between contracted and expanded positions. For example, controlling operation of the actuator to manipulate the coupling wires (e.g. move the coupling wires, or increase/decrease tension in the coupling wires) may cause a corresponding increase/decrease in the radial footprint of the scaffold 158. That is, as the coupling wires are manipulated, the elements of the scaffold 158 may move to cause expansion of the scaffold 158.

It is to be appreciated that in examples where an actuatable scaffold 158 is provided, the sheath 154 need not be provided. For example, the sheath 154 (and sheath coupling 156) may be removed, leaving the actuatable scaffold 158 and the second window 152. Actuation of the scaffold 158 may be controlled so that the scaffold 158 remains in its collapsed configuration (and thus the porous medium 122 is in its insertion state) during insertion of the tube 100 into the patient. During this insertion, the porous medium 122 may still be aligned with the second window 152. Then, to deploy the porous medium 122, the scaffold 158 may be actuated to move into its expanded configuration, thereby providing movement through the second window 152 for deployment of the porous medium 122 into its treatment state.

The nasogastric tube 100 may comprise a lock for inhibiting reverse movement of the scaffold 158/porous medium 122. For example, the lock may comprise a mechanical element actuatable to inhibit movement of the scaffold 158. The lock may be a portion of the scaffold 158, or it may be a portion of the actuator. For example, once the scaffold 158 has been expanded, the lock may be applied to inhibit any movement of the scaffold 158. This may comprise clamping a component of the scaffold 158 and/or a coupling wire, so that no further movement of the scaffold 158 will occur. As one example, the lock may comprise a movable component of the scaffold 158 which, when moved into a locking position, inhibits any collapsing of the scaffold 158. The lock may be moved by controlling operation of the actuator at a proximal region 110 of the device. The lock may be reversed by moving the lock away from the locking position so that the scaffold 158 may then collapse. The lock may be arranged to inhibit reverse deployment of the scaffold 158/porous medium 122 in response to suction being applied to the porous medium 122.

The sheath 154 may be arranged to facilitate selective deployment of the porous medium 122. The sheath 154 may be arranged to retain the porous medium 122 and scaffold 158 radially inwards when in its closed position (to keep the porous medium 122 in its insertion state). The sheath 154 may then be moved into its open position so that the porous medium 122 and scaffold 158 are aligned with the second window 152, and there is no obstruction to movement through the second window 152. The sheath 154 may provide a selective covering for the second window 152 to control whether or not the porous medium 122 may be deployed from the insertion state into the treatment state. In the example shown in FIGS. 3a and 3b, the tube is arranged so that distal movement of the drainage tube 132 may move the sheath 154 away from the second window 152 to enable deployment of the porous medium 122 into the treatment position. The sheath 154 may be arranged to provide ingress of material into the nasogastric tube 100 when in its closed position. For example, the sheath 154 may be closed during insertion to prevent materials entering the nasogastric tube 100 (e.g. and coming into contact with the porous medium 122 etc.). The sheath 154 may be moved into its open position the tube 100 is in place, and the porous medium 122 may be subsequently deployed by actuating the scaffold 158.

Two examples of operation will now be described. In the first example, the scaffold 158 comprises a self-actuating scaffold 158 which is biased to return to its expanded configuration. In the second example, the scaffold 158 is an actuatable scaffold 158 which is controlled by an actuator at a proximal region 110 of the nasogastric tube 100.

In the first example, the nasogastric tube 100 is inserted into position with the suction region of the tube 100 radially inward of, and aligned with, the treatment area 20 and the distal region 130 of the tube 100 located in an area to be drained. The porous medium 122 is retained within the sheath 154, with the scaffold 158 retained in a compressed configuration biased to expand back to its expanded configuration. The sheath 154 retains the scaffold 158 in this compressed configuration. To deploy the porous medium 122, the suction tube 132 is moved relative to the rest of the tube 100. The movement of the suction tube 132 causes a movement of the sheath 154 into an open position in which the sheath 154 is no longer blocking the second window 152. The radial constraint on the scaffold 158 is thus removed, and so the scaffold 158 expands radially outwards towards its expanded configuration. In so doing, a portion of the scaffold 158 and/or a portion of the porous medium 122 is deployed radially outwards through the second window 152. This deployment moves the outer surface of the porous medium 122 into a position for treatment of the treatment area 20. Once in this position, suction is applied to the porous medium 122. The drainage lumen 134 is used for drainage of fluids inside the patient. Once treatment has finished, the nasogastric tube 100 may then be removed. The tube 100 may be removed with the porous medium 122 in a deployed state. Alternatively, the porous medium 122 may be returned to the insertion state, e.g. by moving the sheath 154 back into its closed position (for example, the sheath 154 may be shaped so that movement of the sheath 154 back towards the window 152 may force the porous medium 122 back into the tube, such as by having a tapered inner surface).

In the second example, the tube 100 is inserted into position with the porous medium 122 aligned with the treatment area 20 and the scaffold 158 in a collapsed configuration. To deploy the porous medium 122, the scaffold 158 is actuated to expand radially outwards. In turn, this causes a portion of the scaffold 158 and/or a portion of the porous medium 122 to move radially outwards through the second window 152, so that the porous medium 122 is arranged for treatment. This process may include moving the sheath 154 from a closed state into an open state, or the sheath 154 may not be provided. Once the scaffold 158 is expanded with the porous medium 122 in place for treatment, the lock is applied to inhibit reverse deployment of the scaffold 158. Suction is then applied to the porous medium 122. To remove the tube 100, the lock may be opened, and the scaffold 158 actuated to return to the insertion position. Alternatively, the tube 100 may be removed with the porous medium 122 in the treatment state.

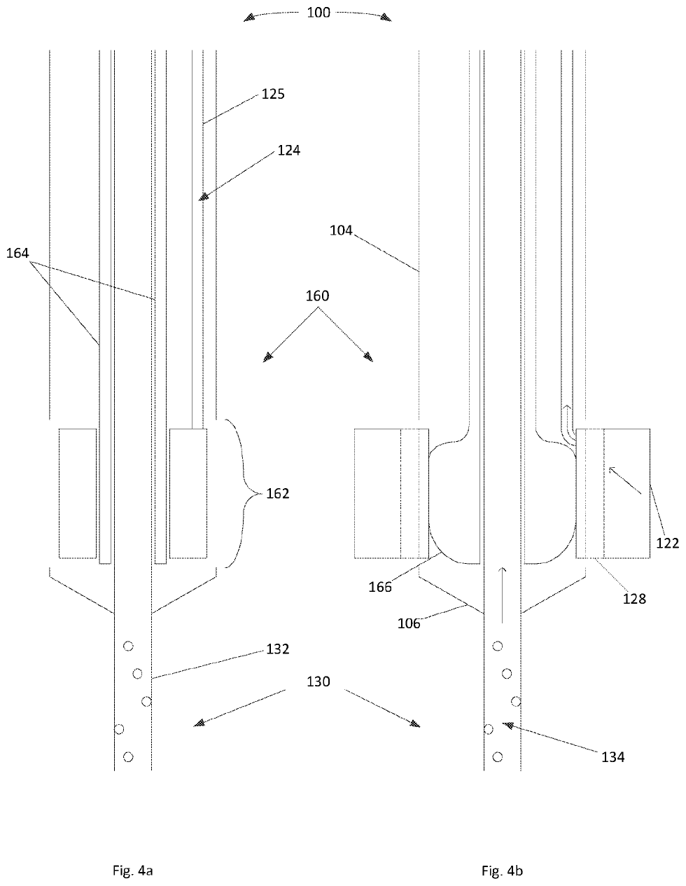

Another example of a nasogastric tube 100 will now be described with reference to FIGS. 4a and 4b. Some of the features of this exemplary nasogastric tube 100 may be similar to those described above with reference to FIGS. 2a, 2b, 3a, and 3b, and so these features shall not be described again. In FIG. 4b, the line arrows indicate flow of fluid.

FIGS. 4a and 4b show cross-section views of a nasogastric tube 100 including a porous medium 122. In FIG. 2a, the porous medium 122 is in an insertion state and in FIG. 2b, the porous medium 122 has been deployed into a treatment state.

The nasogastric tube 100 includes a third radial deployment system 160. The third radial deployment system 160 includes a third window 162 and a balloon 166. The third radial deployment system 160 may also include a balloon lumen 164.

The nasogastric tube 100 includes a suction lumen 124 and a drainage lumen 134. As with FIGS. 3a and 3b, the nasogastric tube 100 includes a suction tube 125, which defines the suction lumen 124. In this example, the suction tube is not annular, and instead it may be cylindrical. The nasogastric tube includes a drainage tube 132, which defines the drainage lumen 134. The nasogastric tube 100 also includes a housing 104. The housing 104 houses the drainage lumen 134, the suction lumen 124, and the balloon lumen 164. The nasogastric tube 100 thus defines at least three separate lumens. In this sense, the housing 104 houses three separate tubes/lumens. A channel 128 is provided in the porous medium 122. The housing 104 includes a distal tip 106.

As with the other FIGS., a distal region 130 of the tube 100 is shown in FIGS. 4a and 4b. Although not shown, the top of the tube 100 in FIGS. 4a and 4b extends in a proximal direction to a proximal region 110 of the tube 100. The porous medium 122 and third radial deployment system 160 are arranged in a suction region 120 of the tube 100. The tube 100 extends beyond the suction region 120 to the distal region 130, such that the two regions are longitudinally offset from each other.

The balloon lumen 164 is coupled to the balloon 166. The balloon 166 is located in the suction region 120 of the nasogastric tube 100. The balloon 166 is positioned radially inward of the porous medium 122. The balloon lumen 164 may extend from the balloon 166 to the proximal region 110 for connection to a source of fluid for inflation of the balloon 166. The balloon 166 and porous medium 122 are located radially inward of the third window 162. In the example shown in FIGS. 4a and 4b, the balloon lumen 164 is annular and circumscribes the drainage lumen 134. However, this need not be the case, as the balloon lumen 164 may be provided by a cylindrical lumen, e.g. which is arranged radially outwards of the drainage lumen 134. In the example of FIGS. 4a and 4b, the suction lumen 124 is provided by a single, non-annular, lumen. The suction lumen 124 is coupled to the porous medium 122 in the suction region 120 of the nasogastric tube 100. The balloon 166 is coupled to the porous medium 122 (e.g. the two may be attached) so that they move together. In other words, the balloon 166 may be coupled to the porous medium 122 so that radial expansion of the balloon 166 (e.g. in response to inflation) may cause the porous medium 122 to also move radially outwards, and vice-versa (e.g. contraction of balloon 126 causes movement of the porous medium 122 radially inward).

The channel 128 extends longitudinally through the porous medium 122. The channel 128 provides a fluid flow path between a region of the body lumen on the proximal side of the porous medium 122 and a region of the body lumen on the distal side of the porous medium 122. A plurality of such channels may be provided. For example, a channel 128 is shown on either side of the porous medium 122 in FIG. 4b. Although only shown in FIG. 4b, it is to be appreciated that such a channel may be provided in the porous medium of any of the examples. For example, a longitudinal channel may extend through the porous medium of FIG. 3b, such that when the scaffold has radially deployed the porous medium, a channel may be defined through the radially deployed porous medium. Similar comments apply to the porous medium shown in FIG. 2b also. Such channels may take any suitable form to enable a fluid flow path for liquids travelling from a proximal side of the deployed porous medium to a distal side (e.g. for liquid drank by a patient).

The housing 104 ends with a distal tip 106. The distal tip 106 may be tapered (e.g. so that it gets narrower the more distally it extends). The third window 162 may separate a main body of the housing 104 from the distal tip 106. The housing 104 circumscribes the drainage lumen 134, the suction lumen 124, and the balloon lumen 164. A coupling at the proximal region 110 of the tube 100 may couple the drainage lumen 134 to an active or passive drainage system, the suction lumen 124 to a source of negative pressure and the balloon lumen 164 to a fluid supply system. In the example of FIGS. 4a and 4b, no covering is provided for the third window 162.

The balloon 166 is expandable. The balloon 166 is arranged so that an outer surface of the balloon 166 will expand radially outwards in response to the balloon 166 being filled with fluid (e.g. a liquid or a gas). The balloon 166 is arranged to move the porous medium 122 radially outward in response to being expanded itself. For example, the balloon 166 may be arranged to expand radially outward as it is inflated (e.g. as it is filled with fluid), and the outer surface of the balloon 166 will contact an inner surface of the porous medium 122 to move the porous medium 122 radially outwards with the balloon 166. The porous medium 122 and balloon 166 are arranged so that this radially outward movement extends through the third window 162 to deploy the porous medium 122 into the treatment state. Deflation of the balloon 166 (e.g. removing fluid from inside the balloon 166) may cause the balloon 166 to shrink, and e.g. for the porous medium 122 to move back radially inwards with the balloon 166 (e.g. to reverse deploy the porous medium 122).

The channel 128 may be arranged to enable fluid to flow from an upstream position (e.g. on a proximal side of the porous medium 122) through the channel 128 to a downstream position (e.g. on a distal side of the porous medium 122). For example, the channel 128 may be arranged to enable liquids consumed by the patient (such as water) to pass through the porous medium 122 (when in the treatment state), e.g. on towards their stomach etc. An inner lining of the channel 128 may be impermeable to fluids. Alternatively, it may not, and such liquid may be absorbed by the porous medium 122. Typically, the patient will be provided with an IV drip, and so will not need to consume liquids (these may just be consumed to alleviate a dry mouth etc.).

The distal tip 106 may be arranged so that it is only the drainage tube 132/drainage lumen which extends distally beyond the suction region 120 of the nasogastric tube 100. In other words, the distal tip 106 may taper down to the drainage tube 132 in the suction region 120 of the tube 100. The distal tip 106 may expand from a narrow radial configuration at its connection point to the drainage tube 132 up to a wider radial configuration near to the porous medium 122 and balloon 166.

In operation, the nasogastric tube 100 is positioned in the patient with the porous medium 122/third window 162 aligned with, and radially within, the treatment area 20. While inserting the tube 100 into the patient and positioning the tube 100 in place, the balloon 166 is not in its expanded state, and so the porous medium 122 is in its insertion state. In this state, the porous medium 122 and the balloon 166 may be located radially inwards of the housing 104. To deploy the porous medium 122, the balloon 166 is controllably filled with a fluid to cause expansion of the balloon 166. As the balloon 166 expands, it will push the porous medium 122 radially outwards. The porous medium 122 may move radially outwards through the third window 162 into position for treatment of the treatment area 20. The balloon lumen 164 may be clamped, or the fluid supply for the balloon 166 may be held constant, to retain the balloon at the same level of inflation (e.g. so that the balloon 166 is at the same volume). The porous medium 122 may then remain in contact with the treatment area 20. To remove the tube 100, the balloon 166 may be drained of fluid so that it shrinks an in so doing brings the porous medium 122 radially inwards (e.g. into the insertion position).

It is to be appreciated in the context of the present disclosure that the above description and associated figures are not to be considered limiting. Instead, this is intended to describe exemplary features which may be used in nasogastric tubes of the present disclosure. For example, in the above examples a drainage tube 132/drainage lumen 134 is provided to enable drainage from the patient simultaneously with application of suction to the porous medium 122. However, this need not be considered limiting. For example, the drainage lumen may not be provided, and instead the tube 100 may just have a porous medium 122 and radial deployment system configured to provide movement radially outwards through a window to radially deploy the porous medium 122 for treatment. In other examples, the drainage lumen 134 may be coupled to the suction lumen 124, to provide an extension thereto. For example, a coupling may be provided at the suction region 120 so that a fluid flow path extends beyond the suction region 120 into a drainage lumen 134.

It is to be appreciated that nasogastric tubes of the present disclosure will typically comprise elongate tubes. A longitudinal axis of such tube may be defined by the central axis running along the length of the tube. The term radially outward need not require the components to be circular, instead, it will be appreciated that this may indicate that features are located further away from this longitudinal axis (e.g. located further away from the centre of the tube).

In examples described herein, a window is provided in a body of the nasogastric tube 100. The window may extend 360 degrees around the tube 100 (or it may extend less). A radial deployment system is provided to controllably provide radial deployment through the window to move an outer surface of the porous medium 122 radially outwards into contact with a treatment area 20 on an internal surface of a body lumen of a patient to be treated. It is to be appreciated that an expander of the deployment system may move radially outwards through the window and/or the porous medium 122 may move radially outwards through the window. For example, a portion of the expander which pushes the porous medium 122 radially outwards (such as a balloon or scaffold) may itself move through the window. In some examples, the porous medium 122 may be entirely located radially outward of the window, and it may only be movement of the radial expander through the window (to provide deployment of the porous medium 122). For example, a window covering may be provided which may be selectively removed to enable the radial expander to move out through the window and then move the porous medium 122 into contact with the treatment area 20. It will also be appreciated that additional movement to enable deployment radially outwards through a window may be envisaged. For example, the suction lumen 124/porous medium 122/radially expander may move proximally or distally within the nasogastric tube 100 to align them with the radial window for radial deployment outwards through the window.

As described above, a radial deployment system may be provided which is configured to cause the porous medium 122 to expand radially outwards through a window in the tube 100. Prior to radial deployment, a constraint may be used to inhibit (unintentional) radial deployment of the porous medium 122. For example, the porous medium 122 itself may be biased to expand radially outward in response to such a constraint being removed. The porous medium may be formed of a self-expanding material which can be compressed, but which will be biased to revert back to an expanded configuration (e.g. of the type shown in FIGS. 2a and 2b). Therefore, once the radial constraint has been removed from that compressed porous medium 122, the medium 122 may expand to be restored to its expanded configuration (as shown in FIG. 2b).

The porous medium 122 may be shaped to facilitate such radial compression and expansion. In particular, the porous medium 122 may be arranged to be radially compressed and longitudinally extended. That is, the porous medium 122 may be configured to increase its length longitudinally in response to being compressed radially (and vice-versa). For example, the porous medium may be arranged to be compressible from: (i) a shorter, wider, configuration where the porous medium 122 spans a larger radial extent, but a shorter longitudinal extent, to (ii) a longer, thinner, configuration where the porous medium 122 spans a narrower radial extent, but a greater longitudinal extent, and vice-versa (e.g. the porous medium may be expandable from the second, narrower, configuration to the first, wider, configuration). For example, the porous medium 122 may be wound radially, e.g. in a helical pattern, such as to form a coil. That porous medium 122 may be biased towards its shorter, wider configuration, where the longitudinal extent of the porous medium 122 is relatively short, but it has a larger radial extent (e.g. a greater diameter), as compared to its compressed configuration.

The tube 100 may be arranged so that such a porous medium 122 may be used to provide the biasing force radially outwards for radial deployment. That is, the tube 100 may be configured so that, in the insertion state, the porous medium 122 is in its long and narrow configuration. The porous medium 122 may then transition to its short and wide configuration to provide the radial deployment of that porous medium 122. For example, the tube 100 may be arranged so that, in response to a radial constraint being removed from the porous medium 122, that porous medium 122 may return to its shorter, wider configuration, thereby to provide the radial deployment of the porous medium 122 (e.g. as the porous medium 122 shrinks in length and expands in width in response to its radial constraint being removed).

The tube 100 may be arranged to facilitate such compression and expansion of the porous medium 122. For this, the nasogastric tube 100 may be arranged with a tube inside a tube (e.g. a tube inside the housing 104). For example, an inner tube, such as drainage tube 132, could be provided inside (radially inward of) the housing 104. These two tubes may be arranged to move in relation to each other, e.g. the housing 104 may be moved over (e.g. longitudinally relative to) the inner tube (drainage tube 132). The porous medium 122 may be coupled to both of these tubes. One portion of the porous medium 122 may be coupled to an outside surface of the inner tube (e.g. drainage tube 132). Another portion of the porous medium 122 may be coupled to an inner surface of the outer tube (e.g. the housing 104). For example, a distal end of the porous medium 122 may be coupled to a point on the outside surface of the drainage tube 132, and a proximal end of the porous medium 122 may be coupled to a point on the inside end of the housing 104. The porous medium 122 may be coupled to the housing 104 at a distal end thereof. The volume between the outer surface of the inner tube and the inner surface of the outer tube (e.g. the volume inside the housing 104 and outside the drainage tube 132) may provide the suction lumen. That is, suction may be applied through that volume to the porous medium (which is held in place in that volume).

The nasogastric tube 100 may be configured so that movement of the housing 104 relative to the drainage tube 132 may vary the longitudinal extent of the porous medium 122. For example, relative movement of one tube in one direction relative to the other tube may cause the porous medium 122 to radially compress into its long and narrow configuration (e.g. for insertion). Relative movement in an opposite direction may reduce a longitudinal extent of the porous medium 122, thereby to cause the porous medium 122 to radially expand into its shorter, wider configuration (e.g. for treatment). For example, the porous medium 122 may be coil shaped, and it may be forced into a long, narrow (and drawn out) coil shape by moving the additional tube in one direction relative to the tube 100, and the porous medium 122 may be forced into a shorter, wider coil by moving the additional tube in the opposite direction relative to the tube 100. The porous medium 122 may be any other suitable shape to facilitate this transition between a radially compressed, longitudinally extended state and a radially expanded, longitudinally compressed state. For example, the porous medium 122 may be arranged to concertina. For example, the porous medium may include a saw tooth-type arrangement for concertinaing. Other example shapes could also be used which have different cross-sections, e.g. circular shapes, or polygonal shapes (triangular, rectangular, square etc.).

In other words, the radial deployment system may comprise a component (e.g. a tube) which is coupled to the porous medium 122. The porous medium 122 may be configured so that movement of this additional component in a first direction (e.g. with respect to the housing 104 of the tube 100) may cause radial compression (and longitudinal extension) of the porous medium 122 (e.g. for providing the porous medium 122 in an insertion state). The porous medium 122 may be configured so that movement of this additional component in a second direction opposite to the first direction (e.g. with respect to the housing of the tube 100) may cause radial expansion (and longitudinal compression) of the porous medium 122 (e.g. for providing the porous medium 122 in a treatment state). For example, in response to this movement in the second direction, the porous medium 122 may be configured to expand radially outward to be deployed for treating a portion of the inner surface of the body lumen to be treated (e.g. an anastomosis).

For example, the porous medium 122 may extend between a distal end of the housing 104 and a more distal region on the drainage tube 132. In the insertion state, the majority of the porous medium 122 (e.g. all of the porous medium 122) may be contained radially inward of the housing 104 (and radially outward of drainage tube 132). As the housing 104 is moved distally relative to the drainage tube 132, the length of porous medium 122 will be shortened, thereby causing the porous medium 122 to expand radially outwards. In other words, a window may be defined between a distal end of the housing 104 (to which the porous medium 122 is coupled) and the portion of the drainage tube 132 to which the porous medium 122 is coupled. Movement of the tubes relative to each other may shorten the length of this separation distance and cause the porous medium 122 to move radially outwards (e.g. through that window) into a treatment state (for treatment).

It is to be appreciated that the particular arrangement of lumens need not be considered limiting. For example, lumens may be annular (e.g. and concentrically arranged) or they may not. It will be appreciated that each of the lumens described herein may be provided by a relevant arrangement of tubes of the nasogastric tube. For example, each lumen may be provided by a region radially inward of a tube of the nasogastric tube. For example, there may be several separate tubes provided and each of these tubes may define a respective lumen. The suction lumen 124/a tube which provides the suction lumen (e.g. suction tube 125) may be located radially outwards from the centre. For example, the suction lumen 124 may be located near to an outer surface of the tube 100, so that the suction lumen 124 will remain coupled to the porous medium 122 after expansion (e.g. if a portion of the porous medium 122 remains radially within the nasogastric tube 100 after deployment). In other examples, the suction lumen 124 (and suction tube 125) may be arranged to move with the porous medium radially outwards.

It is to be appreciated that the particular form and type of porous medium 122 is not to be considered limiting. The porous medium 122 may comprise a material configured to expand into an expanded (treatment) state in response to a radial constraint on the porous medium being removed (e.g. the porous medium 122 may comprise an expandable mesh and/or a foam). The porous medium 122 may comprise material which has been packaged into a certain configuration (e.g. rolled up and/or folded), so that the porous medium 122 may unpackage itself (e.g. unroll/unfold) into an expanded (treatment) state in response to a radial constraint being removed from the porous medium 122 (e.g. the porous medium 122 may comprise a foam, a mesh, a bio-active material and/or an open-pore drainage film). The porous medium 122 may comprise material which is arranged to be expanded in response to expansion of a portion of a radial deployment system arranged internally of the porous medium to radially deploy the porous medium 122 (e.g. the porous medium 122 may comprise a foam, a mesh, a bio-active material and/or an open-pore drainage film). For example, where the radial deployment system comprises a component for radially deploying the porous medium 122, an open-pore drainage film may be used (e.g. as it may provide a thinner material when deployed). Examples of such open-pore drainage films include those made by Suprasorb CNP, Drainage Film; Lohmann & Rauscher International Gmbh & Co.KG, Rengsdorf, Germany.

In the examples shown in FIGS. 2 to 4, the porous medium 122 is generally shown in a cylindrical form. For instance, in each of FIGS. 2b, 3b and 4b, the porous medium appears to have a substantially constant diameter in its radially expanded configuration. However, this should not be considered limiting. Nasogastric tubes 100 of the present disclosure may be configured to facilitate radial deployment of a porous medium 122 that is not uniform in diameter once deployed (and in a treatment state). In particular, nasogastric tubes 100 of the present disclosure may be arranged to provide the porous medium 122, in its deployed configuration, with proximal and distal ends of the porous medium 122 being deployed to a greater radial extent than a central portion of the porous medium 122 (which extends between the proximal and distal end of the porous medium 122).

The nasogastric tube 100 may be configured so that, once deployed, the porous medium 122 is at a greater diameter at its proximal and distal ends than in the central region of the porous medium 122. For example, the force applied radially outwards by the porous medium 122 to the internal surface of the body lumen to be treated may be greater at the distal and proximal ends of the porous medium 122 (e.g. due to the diameter being greater in those regions). Such an arrangement may facilitate greater traction between the porous medium 122 in those regions and the internal surface of the body lumen to be treated. This may help to provide anchoring of the porous medium 122 in place in the body lumen. For example, the tube 100 may be configured to be inserted, and the porous medium 122 radially deployed, so that the proximal and distal ends of the porous medium 122 (where the greater diameter of porous medium 122 engages more with the internal surface of the body lumen) are located either side of the portion of the body lumen to be treated (e.g. either side of an anastomosis). A central region of the porous medium 122 (with smaller diameter) may arranged to be aligned with the treatment area. This may enable the greater diameter areas to help anchor the porous medium in place, without the porous medium 122 exerting a high force radially outwards on the area to be treated. For example, the central region of the porous medium 122 may be slightly narrower than the internal lumen in the region to be treated, but in response to application of suction through the porous medium 122, the region to be treated may be drawn into contact with the central region of the porous medium 122. Such an arrangement may find particular utility for use of the nasogastric tube 100 in a lower GI tract.

The nasogastric tube 100 may be configured to provide this deployed shape (e.g. an hourglass shape) for the porous medium 122 (with a narrower central region, and wider proximal and distal regions). This may be a shape of the porous medium 122 itself. That way, the porous medium 122 may adopt this hourglass shape when in its expanded state (e.g. without requiring additional forces to provide this shape). Additionally, or alternatively, the shape may be at least partially employed due to one or more components of the radial deployment mechanism. For example, where a balloon is expanded to deploy the porous medium, that balloon may be arranged (e.g. shaped) so that, once inflated, it imparts this shape to the porous medium 122. As another example, where a scaffold is used to expand the porous medium 122, that scaffold may be arranged (e.g. shaped) so that it expands the proximal and distal ends of the porous medium 122 to a wider radial configuration than the central region of the porous medium 122. For example, the radial expansion mechanism may be configured to expand the porous medium 122 so that the porous medium 122 will exert a greater force radially outwards at the proximal and distal ends of the porous medium 122 (rather than in its central region of the porous medium 122). That is, above and/or below the area to be treated in the body lumen (e.g. where the proximal and distal ends of the porous medium 122 are located), the porous medium 122 will exert a greater radially outward force onto that body lumen. The central region of the porous medium 122 may be aligned with the area of the body lumen to be treated, and will exert a less great radially outward force in that region of the body lumen.

It will be appreciated from the discussion above that the examples shown in the figures are merely exemplary, and include features which may be generalised, removed or replaced as described herein and as set out in the claims. As will be appreciated by the skilled reader in the context of the present disclosure, each of the examples described herein may be implemented in a variety of different ways. Any feature of any aspects of the disclosure may be combined with any of the other aspects of the disclosure. For example, method aspects may be combined with apparatus aspects, and features described with reference to the operation of particular elements of apparatus may be provided in methods which do not use those particular types of apparatus. In addition, each of the features of each of the examples is intended to be separable from the features which it is described in combination with, unless it is expressly stated that some other feature is essential to its operation. Each of these separable features may of course be combined with any of the other features of the examples in which it is described, or with any of the other features or combination of features of any of the other examples described herein. Furthermore, equivalents and modifications not described above may also be employed without departing from the invention.

Other examples and variations of the disclosure will be apparent to the skilled addressee in the context of the present disclosure.

1. A nasogastric tube comprising:
a porous medium for treatment of a treatment area of an internal surface of a body lumen of a patient to be treated;
a suction lumen coupled to the porous medium for providing suction to the porous medium; and
a radial deployment system operable to deploy the porous medium between:
(i) an insertion state for insertion of the porous medium into the patient, and
(ii) a treatment state in which an outer surface of the porous medium is moved towards the treatment area of the patient;
wherein a window is defined in a body carried by the nasogastric tube; and
wherein a portion of the radial deployment system and/or a portion of the porous medium is arranged to move radially outwards through the window to deploy the porous medium from the insertion state to the treatment state.

2. The nasogastric tube of claim 1, further comprising a drainage lumen extending distally beyond the porous medium for providing drainage of fluid from inside the patient distal to the treatment area.

3. The nasogastric tube of claim 1, wherein the radial deployment system comprises at least one of:
a radial expander arranged to move the porous medium radially outwards to deploy the porous medium from the insertion state to the treatment state; and
a removable covering arranged to inhibit movement of the portion of the radial deployment system and/or the portion of the porous medium radially outwards through the window to deploy the porous medium from the insertion state to the treatment state.

4. The nasogastric tube of claim 3, wherein the radial expander comprises a scaffold actuatable to move from a contracted configuration to an expanded configuration to deploy the porous medium from the insertion state into the treatment state.

5. The nasogastric tube of claim 4, wherein the scaffold is arranged to be positioned radially within the porous medium and to expand radially outward when moving from the contracted configuration to the expanded configuration.

6. The nasogastric tube of claim 4, wherein the scaffold comprises one or more mechanical coupling points arranged to enable a wire to be coupled to that coupling point for controlling actuation of the scaffold by manipulating said wire.

7. The nasogastric tube of claim 6, further comprising one or more said wires, wherein each wire is coupled to a coupling point of the scaffold and is manipulatable from outside the patient to control actuation of the scaffold.

8. (canceled)

9. The nasogastric tube of claim 4, wherein the scaffold is made of a shape memory material biased to return to the expanded configuration.

10. The nasogastric tube of claim 3, wherein the radial expander comprises a balloon expandable to deploy the porous medium from the insertion state into the treatment state.

11. The nasogastric tube of claim 10, wherein the balloon is arranged to be positioned radially within the porous medium and to expand radially outward to deploy the porous medium from the insertion state into the treatment state.

12. (canceled)

13. The nasogastric tube claim 3, wherein the porous medium is configured to radially expand to deploy the porous medium into the treatment state.

14. (canceled)

15. The nasogastric tube of claim 3, wherein the radial expander comprises a biasing element arranged to bias the porous medium towards the expanded configuration.

16. The nasogastric tube of claim 3, wherein a portion of the radial expander is arranged to move radially outwards through the window to deploy the porous medium from the insertion state into the treatment state.

17. The nasogastric tube of claim 3, wherein the suction lumen is coupled to and/or mechanically supported by a portion of the radial expander.

18. The nasogastric tube of claim 3, wherein the removable covering is arranged to exist in:
a closed state in which movement through the window to deploy the porous medium from the insertion state to the treatment state is inhibited; and
an open state in which movement through the window to deploy the porous medium from the insertion state to the treatment state is enabled.

19. The nasogastric tube of claim 18, wherein the removable covering comprises a sheath operable to move relative to the window of the nasogastric tube between a closed position and an open position to provide the closed and open state respectively.

20. The nasogastric tube of claim 18, wherein the removable covering comprises a dissolvable member configured to dissolve in response to application of a selected fluid thereto.

21. The nasogastric tube of claim 1, further comprising a lock operable to inhibit reverse deployment of the porous medium from the treatment state into the insertion state.

22. (canceled)

23. The nasogastric tube of claim 1, wherein the porous medium is configured to deploy radially outwards through the window about substantially 360 degrees.

24. The nasogastric tube of claim 1, further comprising a housing arranged to house the components of the nasogastric tube, and wherein the window is provided in the housing.

25. (canceled)
